(19) United States
(12) Patent Application Publication
(10) Pub. No.: US 2023/0157992 A1
(43) Pub. Date: May 25, 2023

(54) Novel Cannabinoids Formulations and Their Use for the Treatment of Acute Flaccid Myelitis

(71) Applicant: Diverse Biotech, Inc., Miami, FL (US)

(72) Inventors: Philip Arlen, Miami Beach, FL (US); Stella K. Vnook, Churchville, PA (US)

(73) Assignee: Diverse Biotech, Inc., Miami, FL (US)

(21) Appl. No.: 17/798,171

(22) PCT Filed: Feb. 8, 2021
(86) PCT No.: PCT/US21/17042

§ 371 Date: Aug. 8, 2022

Related U.S. Application Data
(60) Provisional application No. 62/971,956, filed on Feb. 8, 2020.

(51) Int. Cl. A61K 31/352; A61K 31/05; A61K 31/343; A61K 31/192

(52) U.S. Cl. CPC A61K 31/352; A61K 31/05; A61K 31/343; A61K 31/192

(57) ABSTRACT

The present disclosure describes methods of treating acute flaccid myelitis (AFM) with one or more cannabinoids, as well as formulations and composition containing one or more cannabinoids and at least on excipient and their use for the treatment of AFM.

### FIELD OF INVENTION

The present invention describes the use of cannabinoids for the treatment of acute flaccid myelitis (AFM). The present invention also describes cannabinoid containing formulations and their method of use for the treatment of AFM. The present invention also describes formulations containing at least one cannabinoid that contain at least one excipient and their method of use for the treatment of AFM.

### BACKGROUND OF THE INVENTION

Acute flaccid myelitis (AFM) is a neuroinflammatory disease that is characterized by flaccid paralysis of one or more limbs, symptoms that often appear after a viral illness. The disease affects the spinal cord gray matter, resulting in limpness and weakness in affected muscles. Magnetic resonance imaging (MRI) findings are consistent with inflammation of the spinal cord gray matter (Fatemi and Chakraborty 2019). Sadly, once symptoms present, the long-term outcomes are poor as many patients with AFM never fully recover neurologic function. In the severest cases, extensive myelitis can lead to respiratory failure and death (Andersen et al. 2017).

The cause of AFM is somewhat controversial. Current thinking has non-polio enteroviruses, particularly D68 and A71, as the etiological cause of AFM, given epidemiological evidence supports a temporal association between respiratory enteroviral illness and clustering of AFM cases (Fatemi and Chakraborty 2019). Since the elimination of wild-type poliovirus from the Western Hemisphere in 1991 (Centers for Disease Control and Prevention (CDC) 1994), other causative agents have been implicated, including flaviviruses such as West Nile and Japanese encephalitis viruses (Sejvar et al. 2016, Solomon et al. 1998), herpesviruses (Wong, Connolly, and Noetzel 1999), and adenoviruses (Ivanova et al. 2012). At a cellular or molecular level, the pathogenesis of AFM remains unclear but the clinical presentation may be due to post-infectious immune responses to these viruses with accompanying inflammatory damage to gray matter (Messacar et al. 2015, Yea et al. 2017).

Treatments for AFM are primarily supportive and immunomodulatory in nature, and include adjunctive therapies such as intravenous immunoglobulin (IVIG), pulse therapy with corticosteroids, plasmapheresis, and fluoxetine, none of which improves long-term outcomes. In at least one retrospective cohort study, in which patients received treatment for AFM that included IVIG and steroids, with more severe patients also receiving plasmapheresis and/or fluoxetine, no correlation was seen between treatment received, timing of treatment, order of treatment, and recovery outcome (Kane et al. 2019). Consequently, new treatment options are required to address this severe but poorly understood illness.

Separately, there are more than 140 different cannabinoids found in the plant Cannabis sativa L. The most widely recognized cannabinoid is Δ9-tetrahydrocannabinol (THC); second is cannabidiol (CBD), which, unlike THC, possesses no psychoactivity. The literature is replete with studies demonstrating the benefits of cannabinoids, including CBD: against cancer, anxiety, psychosis, convulsive behavior, and, inflammation (Malfitano et al. 2011, Pisanti et al. 2009, Pisanti et al. 2013, Pertwee 2004).

The endocannabinoid system (ECS), a network of enzymes, receptors, and signal mediators, has been implicated in regulating both peripheral and neuroinflammation via its effects on the immune system (Mackie and Stella 2006). Neuroinflammation is of particular risk given the consequences: prolonged exposure to inflammatory conditions in the brain has been linked with the development of neurodegenerative diseases such as Alzheimer's disease, Parkinson's disease, and multiple sclerosis (Gordon and Woodruff 2017). The inflammatory microenvironment is also known to be a driving force behind the progression of incipient neoplasias into highly malignant tumors such as glioblastoma (Mantovani et al. 2008, Solinas et al. 2010, Yeung et al. 2013).

Cannabinoids have shown beneficial effects on neuroinflammatory and neurodegenerative processes through various biological targets (reviewed in (Borrelli et al. 2014)), including cannabinoid receptors (CB)1 and CB2 (Granja et al. 2012), transient receptor potential (TRP) channels (De Petrocellis et al. 2011), cyclooxygenase (COX) 1 and COX 2 enzymes (Mantovani et al. 2008), 5-hydroxytryptamine receptor 1A (5-HT1A) and the a2-adrenergic receptor (Cascio et al. 2010), and the nuclear receptor peroxisome proliferator-activated receptor-gamma (PPAR-γ) (Granja et al. 2012).

Both endogenous cannabinoid receptors, CB1 and CB2, are expressed in the nervous system, the latter especially during inflammatory episodes (Maresz et al. 2007). Endocannabinoids impart their neuroprotective effects via the suppression of pro-inflammatory cytokines and increased production of anti-inflammatory cytokines (Pellati et al. 2018). Their plant counterparts derived from C. sativa, the phytocannabinoids, are promising candidates for the management of several neuroinflammatory conditions (Borgonetti et al. 2019). In a rodent model of inflammation, CBD was able to suppress the release of tumor necrosis factor (TNF)-α, IL-1β, and IL-6 in both a CB2-dependent manner (Hunter and Burstein 1997) and via interaction with the adenosine A2A receptor, transient receptor potential (TRP) V1 receptor, G protein-coupled receptor GPR55, and homodimerization of CB2 and the serotonin 1A (5HT1A) receptor (Burstein 2015).

The inflammatory microenvironment associated with AFM has been characterized in at least one patient with the disease (Hidaka et al. 2019), in whom the levels of interleukin (IL)-6 and interferon (IFN)-γ were significantly elevated in the cerebral spinal fluid.

### BRIEF SUMMARY OF THE INVENTION

The present invention is directed toward a method of treating acute flaccid myelitis (AFM) consisting of administering to an individual in need thereof an effective amount of a compound selected from the group consisting of

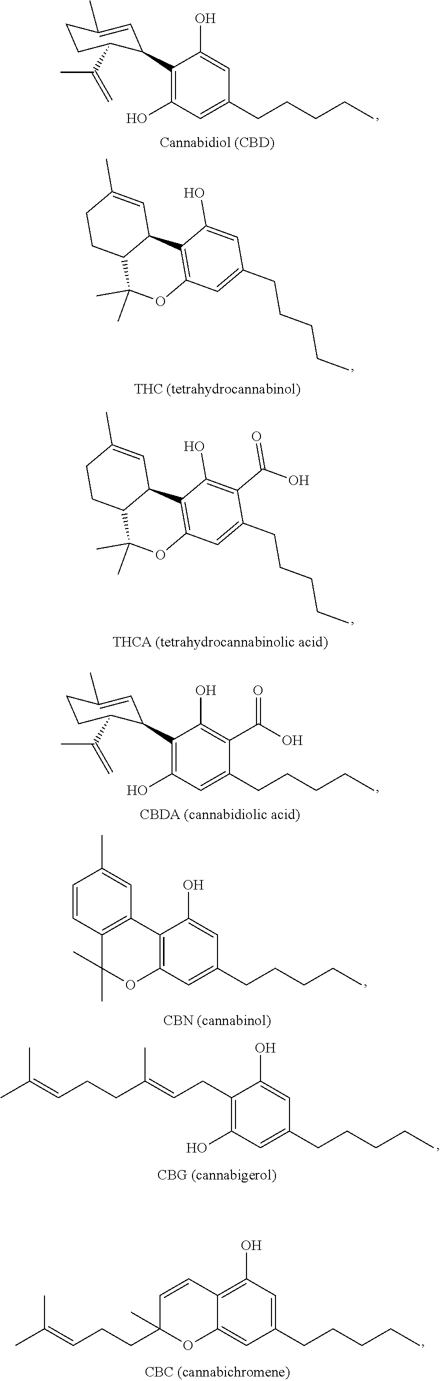

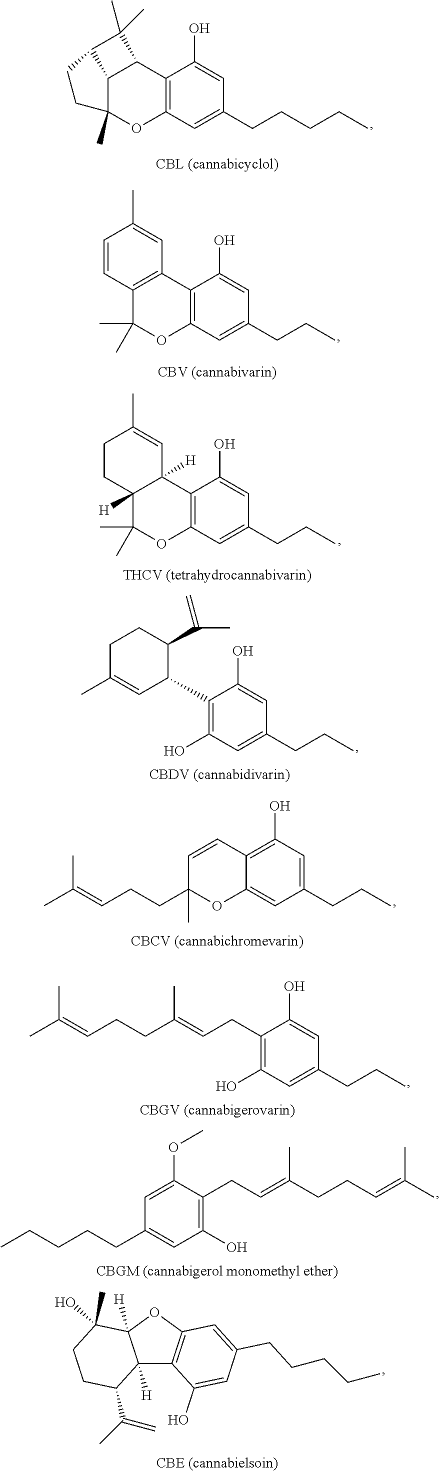

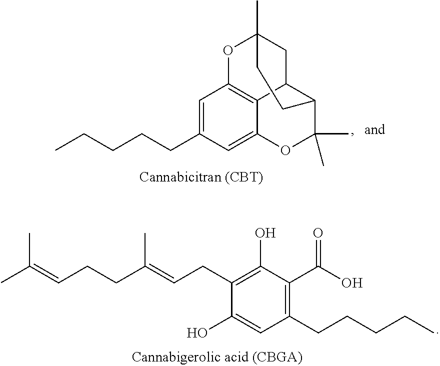

The present invention further relates to compositions and formulations consisting of an effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient.

The present invention yet further relates to a method for treating or preventing AFM, wherein said method comprises administering to a subject a composition consisting of an effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient.

The present invention further relates to compositions and formulations consisting of an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient.

The present invention yet further relates to a method for treating or preventing AFM, wherein said method comprises administering to a subject a composition consisting of an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient.

The present invention also relates to a method for treating or preventing disease or conditions associated with AFM. Said methods comprise administering to a subject an effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA.

The present invention yet further relates to a method for treating or preventing disease or conditions associated with AFM, wherein said method comprises administering to a subject a composition consisting of an effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient.

The present invention also relates to a method for treating or preventing disease or conditions associated with AFM. Said methods comprise administering to a subject an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA.

The present invention yet further relates to a method for treating or preventing disease or conditions associated with AFM, wherein said method comprises administering to a subject a composition consisting of an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient.

The present invention further relates to a process for preparing the composition consisting of an effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient of the present invention.

The present invention further relates to a process for preparing the composition consisting of an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION OF THE INVENTION

Throughout the description, where compositions and formulations are described as having, including, or comprising specific components, or where processes are described as having, including, or comprising specific process steps, it is contemplated that compositions and formulations of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

The terms “treat” and “treating” and “treatment” as used herein, refer to partially or completely alleviating, inhibiting, ameliorating, and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which CBD can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compositions and formulations of the present invention.

### Formulations

The present invention also relates to compositions or formulations which comprise a compound selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient. The present invention further relates to compositions or formulations which comprise a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient. In general, the compositions and formulations of the present invention comprise an effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA; and one or more excipients which are effective for treating or preventing AFM. In some embodiments, the compositions and formulations of the present invention comprise an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA; and at least one excipient which are effective for treating or preventing AFM.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach.

The present teachings also provide pharmaceutical compositions that include compounds of the disclosure and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compositions and formulations of the present teachings can be administered orally or parenterally, or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. Compounds of the disclosure can be formulated in a conventional manner, for example, in a manner similar to that used for known therapeutic agents. Oral formulations containing compounds of the disclosure can comprise any conventionally used oral form, including tablets, capsules, buccal forms, troches, lozenges, and oral liquids, suspensions or solutions. In powders, the carrier can be a finely divided solid, which is an admixture with finely divided compounds of the disclosure. In tablets, compounds of the disclosure can be mixed with a carrier having the necessary compression properties in suitable proportions and compacted in the shape and size desired. The powders and tablets can contain up to 99% of a compound selected from the group consisting of THC, THCA, CBD, CBDA, CBN, CBG, CBC, CBL, CBV, THCV, CBDV, CBCV, CBGV, CBGM, CBE, CBT, and CBGA.

Capsules can contain mixtures of compounds of the disclosure with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of compounds of the disclosure. The oral formulation can also consist of administering compounds of the disclosure in water or fruit juice, containing appropriate solubilizers or emulsifiers as needed.

Liquid carriers can be used in preparing solutions, suspensions, emulsions, syrups, elixirs, and for inhaled delivery. Compounds of the disclosure can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of compounds of the disclosure. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg of compounds of the disclosure to about 1,500 mg of compounds of the disclosure, and can be given in a single dose or in two or more doses. Such doses can be administered in any manner useful in directing compounds of the disclosure to the recipient's bloodstream, including orally, via implants, parenterally (including intravenous, intraperitoneal, intrathecal, and subcutaneous injections), rectally, vaginally, and transdermally.

When administered for the treatment of AFM, it is understood that an effective dosage can vary depending upon the particular composition or formulation utilized, the mode of administration, and severity of the condition being treated, as well as the various physical factors related to the individual being treated. In therapeutic applications, a composition or formulation of the present teachings can be provided to a patient already suffering from AFM in an amount sufficient to cure or at least partially ameliorate the symptoms of AFM and its complications. The dosage to be used in the treatment of a specific individual typically must be subjectively determined by the attending physician. The variables involved include the specific condition and its state as well as the size, age, and response pattern of the patient.

In some cases it may be desirable to administer compounds of the disclosure directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, compounds of the disclosure can be formulated into a liquid composition, a solid composition, or an aerosol composition. The liquid composition can include, by way of illustration, compounds of the disclosure dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including compounds of the disclosure intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, compounds of the disclosure, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds of the disclosure, compositions described herein, and formulations described herein can be administered parenterally or intraperitoneally. Solutions or suspensions of compounds of the disclosure can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical compositions and formulations of compounds of the disclosure suitable for injection can include sterile aqueous solutions or dispersions and sterile powders for the extemporaneous preparation of sterile injectable solutions or dispersions. In some embodiments, the compositions and formulations can be sterile and its viscosity permits it to flow through a syringe. The compositions and formulations preferably are stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds of the disclosure, compositions described herein, and formulations described herein can be administered transdermally, i.e., administered across the surface of the body and the inner linings of bodily passages including epithelial and mucosal tissues. Such administration can be carried out using compounds of the disclosure in lotions, creams, foams, patches, suspensions, solutions, and suppositories (rectal and vaginal).

Transdermal administration can be accomplished through the use of a transdermal patch containing compounds of the disclosure and a carrier that can be inert to compounds of the disclosure, can be non-toxic to the skin, and can allow delivery of compounds of the disclosure for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing compounds of the disclosure can also be suitable. A variety of occlusive devices can be used to release compounds of the disclosure into the blood stream, such as a semi-permeable membrane covering a reservoir containing compounds of the disclosure with or without a carrier, or a matrix containing compounds of the disclosure. Other occlusive devices are known in the literature.

Compositions of compounds of the disclosure and formulations described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the disclosure into host cells either in vitro or in vivo. Lipid formulations and nanocapsules can be prepared by methods known in the art.

To increase the effectiveness of compounds of the disclosure, compositions of the present teachings, and formulations of the present teachings, it can be desirable to combine compounds of the disclosure with other agents effective in the treatment of the target disease. For example, other active compounds (i.e., other active ingredients or agents) effective in treating the target disease can be administered with compounds of the disclosure. The other agents can be administered at the same time or at different times than compounds of the disclosure. Alternatively, other active compounds (i.e., other active ingredients or agents) effective in treating the target disease can be administered with compositions of the present teachings. The other agents can be administered at the same time or at different times than compositions of the present teachings. Alternatively, other active compounds (i.e., other active ingredients or agents) effective in treating the target disease can be administered with formulations of the present teachings. The other agents can be administered at the same time or at different times than formulations of the present teachings.

Compounds of the disclosure, compositions of the present teachings, and formulations of the present teachings can be useful for the treatment AFM in a mammal, for example, a human subject. The present teachings accordingly provide methods of treating AFM by providing to a mammal compounds of the disclosure or a pharmaceutical composition that includes compounds of the disclosure in combination or association with pharmaceutically acceptable carriers. Compounds of the disclosure can be administered alone or in combination with other therapeutically effective compounds or therapies for the treatment AFM.

Non-limiting examples of compositions according to the present invention include from about 0.001 mg to about 1500 mg of compounds of the disclosure and one or more excipients; from about 0.01 mg to about 100 mg of compounds of the disclosure and one or more excipients; and from about 0.1 mg to about 10 mg of compounds of the disclosure; and one or more excipients.

### Procedures

The Examples provided below provide representative methods for preparing compositions containing compounds of the disclosure and formulations of the present invention. The skilled practitioner will know how to substitute the appropriate excipients known to those skilled in the art, in order to prepare the compositions and formulations of the present invention.

Example 1: Extraction and Crystallization of a Cannabinoid Preparation: Hemp cultivars cloned from high CBD-producing mother plants were transplanted into fields and grown under organic conditions without the use of pesticides or herbicides. Hemp was harvested and flash-dried, and a crude extract was obtained using CO₂ as the solvent.

The crude extract was dewaxed using a solvent-assisted precipitation and filtration step to provide a refined extract, which was then placed in a stainless steel crystallization tank. After 24-48 hours, the crystals were washed several times, dried, and ground, resulting in a crystalline cannabinoid preparation in which 99.62% by weight was CBD (Table 1).

Example 2: Preparation of a Pharmaceutical Composition: The cannabinoid preparation obtained in Example 1 was formulated in hempseed oil and analyzed for plant-based cannabinoids by convergence chromatography. The data collected were compared to data collected for certified reference standards at known concentrations.

TABLE 1

| Compound | Weight % | Concentration (mg/g) |
|---|---|---|
| Δ9-THC | ND | ND |
| THCV | ND | ND |
| CBD | 99.62 | 996.20 |
| CBDV | 0.18 | 1.80 |
| CBG | ND | ND |
| CBC | ND | ND |
| THCA | ND | ND |
| CBDA | ND | ND |
| CBGA | ND | ND |
| TOTAL | 99.80 | 998.00 |

Max THC (and Max CBD) were calculated values for total cannabinoids after heating, assuming complete decarboxylation of the acid to the neutral form, i.e., the values were calculated based on the weight loss of the acid group during decarboxylation. Max THC=(0.877×THCA)+THC. ND=None detected above the limits of detection.

The formulation was tested for microbiological contaminants in accordance with the requirements of ISO/IEC 17-25. The results are shown in Table 2.

TABLE 2

| Analysis | Results (CFU/g) | Limit (CFU/g)* |
|---|---|---|
| total aerobic bacterial count | <100 | 10,000 |
| total coliform bacterial count | <100 | 100 |
| total bile tolerant gram negative count | <100 | 100 |
| total yeast and mold | <100 | 1,000 |

*Qualitative calculation based on recorded peak areas.

Finally, the terpene profile of the formulation was analyzed by head-space gas chromatography. The data collected were compared to data collected for certified reference standards at known concentrations. These results are shown in Table 3.

TABLE 3

| Compound | ppm |
|---|---|
| Myrcene | |
| Pulegone | |
| Isopulegol | |
| Borneol | |
| Menthol | |
| nerolidol-cis | |
| γ-terpenine | |
| nerolidol-trans | |
| α-bisabolol | 2 |
| Linalool | 1 |
| linalyl Acetate | |
| β-caryophyllene | 4 |
| carophyllene oxide | 1 |
| Eugenol | |
| Guaiol | 1 |
| Sabinene | |
| Humulene | 1 |
| p-cymene | |
| terpineol | |
| camphene | |
| fenchone | |
| β-pinene | |
| eucalyptol | |
| α-terpenine | |
| 3-carene | |
| α-pinene | |
| citral-1 | |
| citral-2 | |
| limonene | 1 |
| citronellol | |
| geraniol | |
| ocimene-2 | |
| ocimene-1 | |
| α-phellandrene* | 1 |
| terpinolene | |

What is claimed is:

1. A method of treating acute flaccid myelitis (AFM) consisting of administering to an individual in need thereof an effective amount of a compound selected from the group consisting of

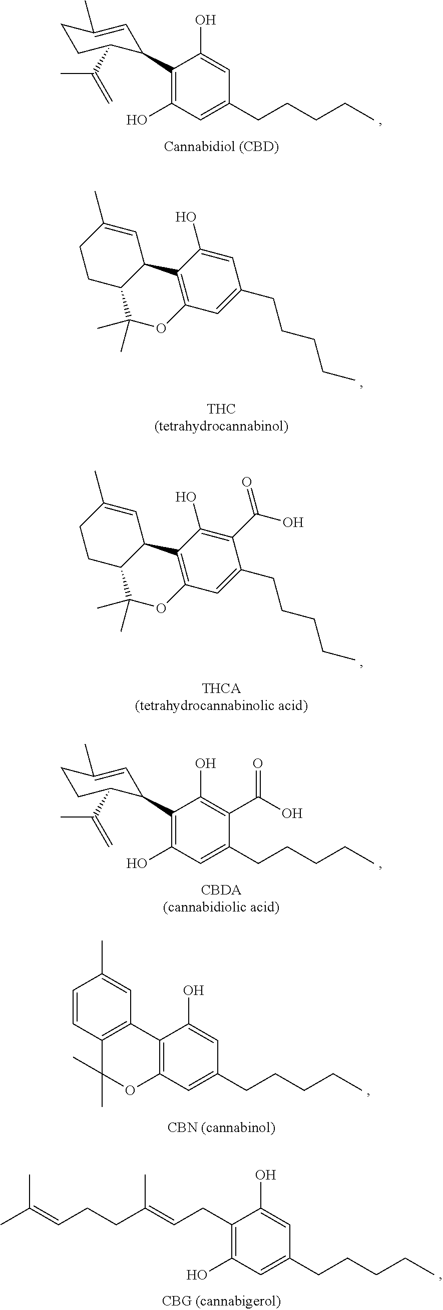

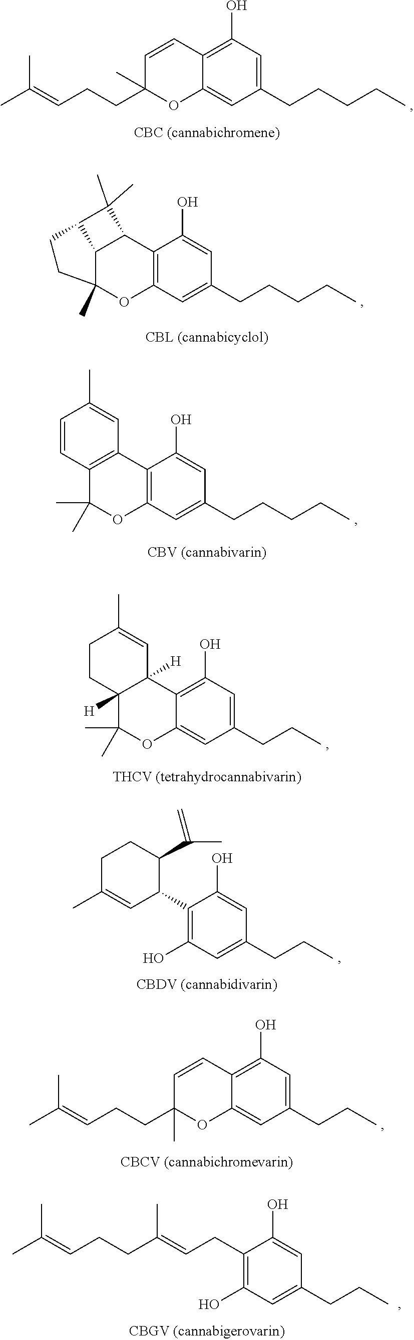

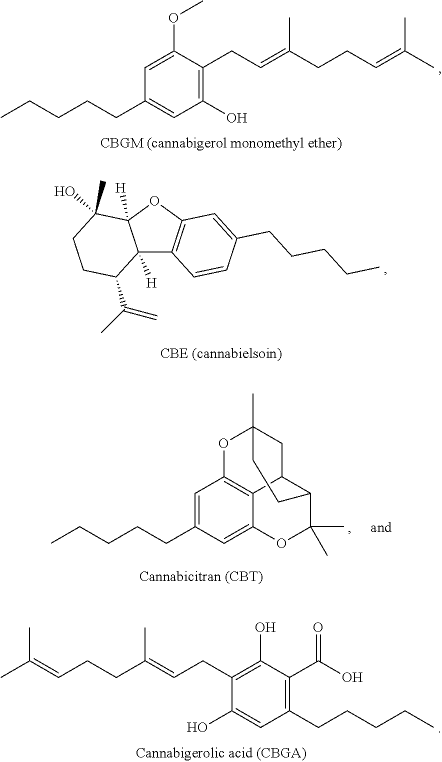

2. The methods of claim 1 wherein the effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA is in a formulation or composition that includes at least one excipient.

3. A method of preventing acute flaccid myelitis (AFM) consisting of administering to an individual in need thereof an effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA.

4. The methods of claim 3 wherein the effective amount of a compound selected from the group consisting of THC, THCA, CBD, CBDA, CBN, CBG, CBC, CBL, CBV, THCV, CBDV, CBCV, CBGV, CBGM, CBE, CBT, and CBGA is in a formulation or composition that includes at least one excipient.

5. A method of treating acute flaccid myelitis (AFM) consisting of administering to an individual in need thereof an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA.

6. The method of claim 5 wherein the mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA is in a formulation or composition that includes at least one excipient.

7. A method of preventing acute flaccid myelitis (AFM) consisting of administering to an individual in need thereof an effective amount of a mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA.

8. The method of claim 7 wherein the mixture of at least two compounds selected from the group consisting of THC, THCA, CBD, CBDA, CBN, CBG, CBC, CBL, CBV, THCV, CBDV, CBCV, CBGV, CBGM, CBE, CBT, and CBGA is in a formulation or composition that includes at least one excipient.
